(19) United States
(12) Patent Application Publication
(10) Pub. No.: US 2022/0415468 A1
(43) Pub. Date: Dec. 29, 2022

(54) Portable Prepackaged Medication Delivery Device

(71) Applicant: David Robert Schoenfeld, Petal, MS (US)

(72) Inventor: David Robert Schoenfeld, Petal, MS (US)

(21) Appl. No.: 17/229,931

(22) Filed: Jun. 29, 2021

(51) Int. Cl. G16H 20/13; G16H 40/63; A61J 7/00

(52) U.S. Cl. CPC G16H 20/13; G16H 40/63; A61J 7/0076

(57) ABSTRACT

This invention relates to an improved means of easily, quickly, and sanitarily distributing prepackaged medication to patients in a hospital or nursing home. The invention utilizes an small easily wiped down and cleaned housing which can be carried by a shoulder strap from patient to patient without leaving it unattended in a hallway. An internal bar-code reader and touch screen computer provide a positive means of identifying each prepackaged medication container, recording patient vital information, presenting to a nurse only the medication for a specific patient, and transmitting vital information and time of dispense to a hospital's inhouse computer network.

### BACKGROUND OF THE INVENTION

Nurses prepare and deliver medications to patients several times per day and spend considerable time doing so. Several methods are currently used; one method is for a nurse to individually prepare then carry the medications from the nurse's station to the patient's room and then return to the nurse's station to get the medications for the next patient. This is currently the most secure and sanitary method. This can be repeated until all the patients on a hospital wing have been given their medications. Another method is to load the medications for all the patients on a hospital wing into a cart and then roll the cart from patient's room to patient's room. Carts with wheels can be sophisticated carts with a large worksurface and computer storage compartment, a full size computer monitor, and a cabinet mounted below the computer storage compartment with numerous bins in which medications can be secured in a variety of arrangements. Other wheeled carts are more like a lockable mechanics roll around tool chest with multiple drawers and doors. There are numerous options for wheeled carts such as these, both with and without computes and various degrees of automation, all of which are heavy and award to push and steer, difficult to effectively sanitize, and somewhat expensive. With any type of cart, this method generally requires less walking, but does require pushing a heavy cart. Although the bins and drawers on the push carts are generally locked, it is not good practice for the nurse to leave the cart unattended outside a patients from, particularly if narcotics are stored in the cart. Moving a large and heavy cart into each patient's room is also a good way to transfer bacteria from one patient to the next. Infection control regulations often dictates that the cart should be disinfected after going into a patient's room.

There is also a trend for pharmacies to prepackage all of a patient's medications for a single pour into a single blister pack or sealed container rather than a nurse carrying a variety of single medications from patient to patient.

This invention further makes use of this trend in medication packaging and facilitates delivering medication to patients' rooms conveniently without the necessity of pushing and steering a heavy cart.

### DETAILED DESCRIPTION OF THE INVENTION

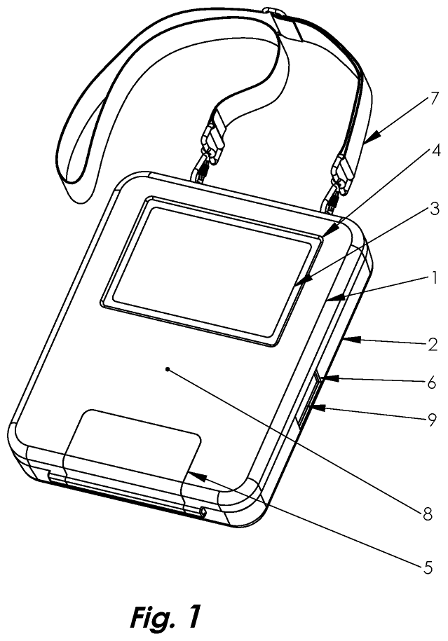

FIG. 1 shows an assembled view of the invention in the secured transport mode. The invention is comprised of an upper enclosure 1 and lower enclosure 2 that comprise a basically hollow box which may be of a rigid plastic material such as ABS and is 10.5×13×2.5 inches high but may be larger or smaller depending on the size, quantity, and configuration of the personal medication containers utilized. A tablet computer 3 is rigidly mounted internally in the general location shown with the touch screen portion of said tablet computer is accessible through a tablet computer opening 4 on said upper enclosure. said tablet computer may be a Best Buy model 15668 or similar size table style computer with a built in touch screen. A load door 5 is said upper enclosure for loading multiple personal medication containers 20 at one time. A dispense door opening 6 in said lower enclosure is blocked by a dispense door 9.

said tablet computer controls the functions of the inner workings of the invention and communicates with a hospital's wireless computer system via built in WiFi. said tablet computer has various screens to allow a nurse enter personal access codes to control the functions of the invention. One screen may allow a nurse to log into the invention, another screen may allow the nurse to open said load door for loading said personal medication containers, another screen may advance the carriage cup chain to make additional cups available for loading, another screen may display the names of the patients' personal medication containers that loaded in the invention, another a screen may allow the nurse to select a patient for the information on the personal medication containers, another screen may allow the nurse to input a patient's vital information and notes for convenient transition via WiFi for storage on the hospital's computer system, another screen may allow a nurse to open said dispense door and dispense said personal medication container for the previously selected patient, another screen may shown an actual image of the various medication that a nurse should expect to find in the personal medication container as a final verification that the correct medications were put in the personal medication container, another screen may allow the nurse to select that the transfer of the medications to the patient is complete and for said dispense door to close and transmit via WiFi the time of delivery to the hospital's computer system.

A writing surface 8 on said upper enclosure adjacent to and below said tablet computer is provided as convenient writing surface as needed by a nurse. A carrying strap 7 is attached to said lower enclosure for easily carrying this device. This is the normal transport mode with both said doors closed and locked and the internal said personal medication containers secured.

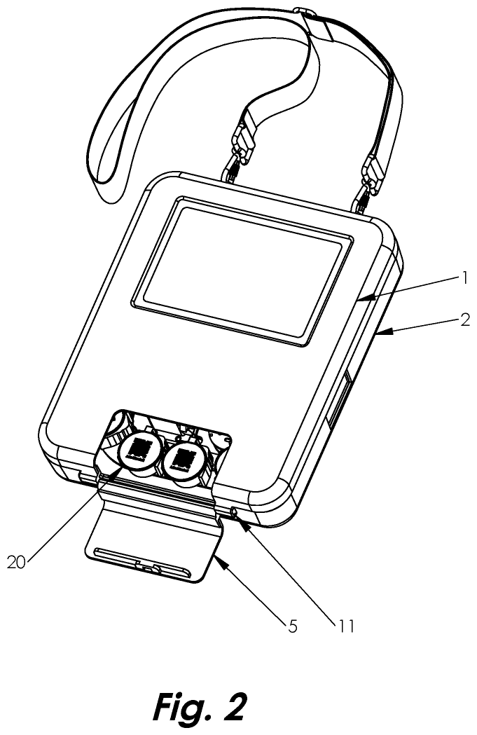

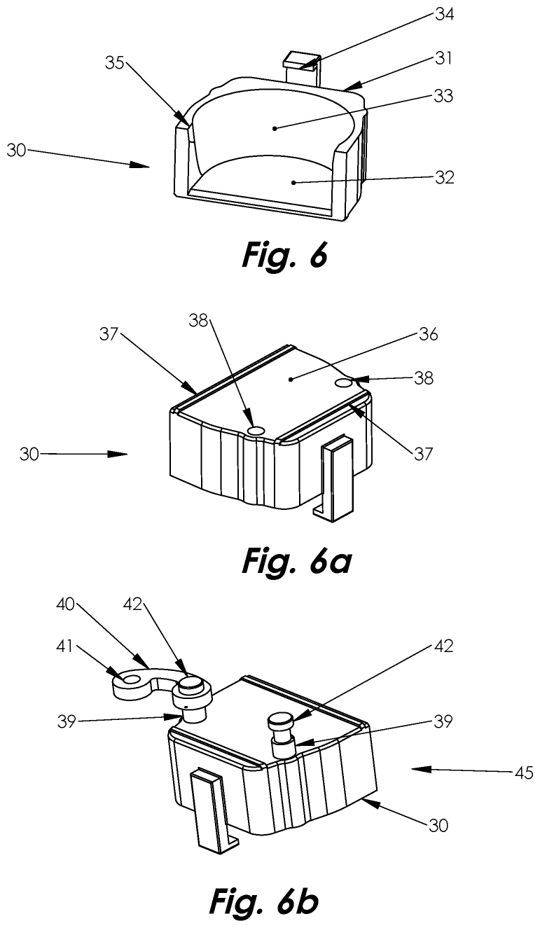

FIG. 2 shows the same view as FIG. 1, but with said load door in its open position with two personal medication containers placed in carriages. Carriages are describe in FIG. 6, FIG. 6a, and FIG. 6b.

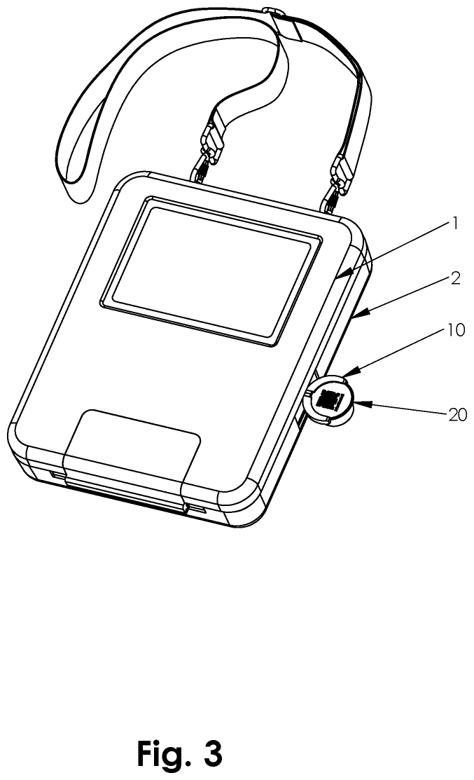

FIG. 3 shows the same view as FIG. 1, but with said dispense door retracted, thereby providing a passage through said lower enclosure via said dispense door opening for the dispense arm 10. Said dispense arm can now pick up a personal medication container 20 and move it to an external position as shown, thereby providing a nurse at a patient's bedside access to single said personal medication container.

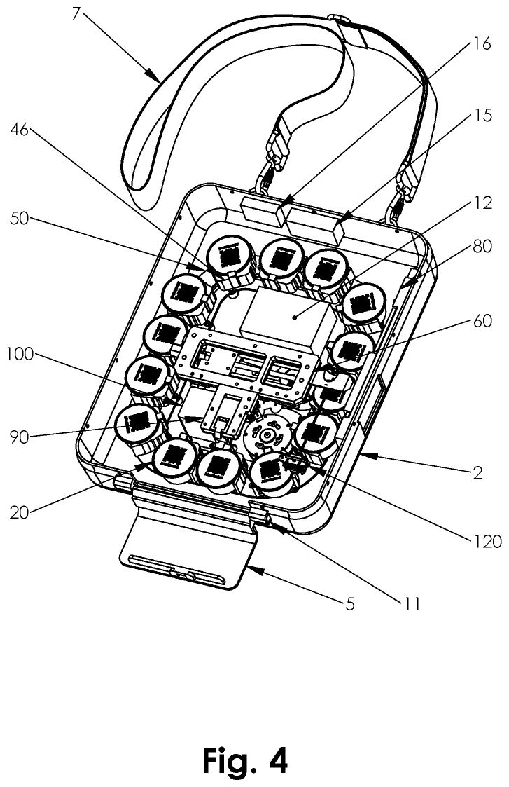

FIG. 4 shows the same view as FIG. 1, but with said upper enclosure and said tablet computer removed. The various modules are the dispense arm module 60, dispense door module 80, load door latch module 90, carriage chain drive module 100, the guide track module 50, and the bar-code reader module 120 are identified and further explained\] in corresponding drawings. Additionally, a battery 12, an electronic tablet computer interface 15, an electronic motor driver 16, and the load door hinge pin 11 are identified.

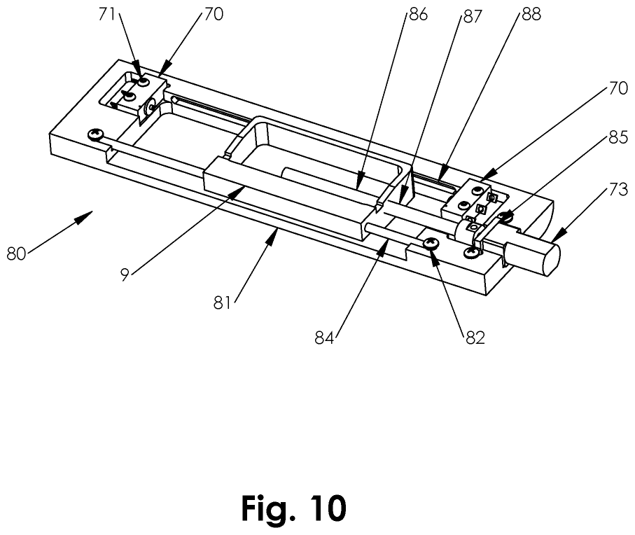

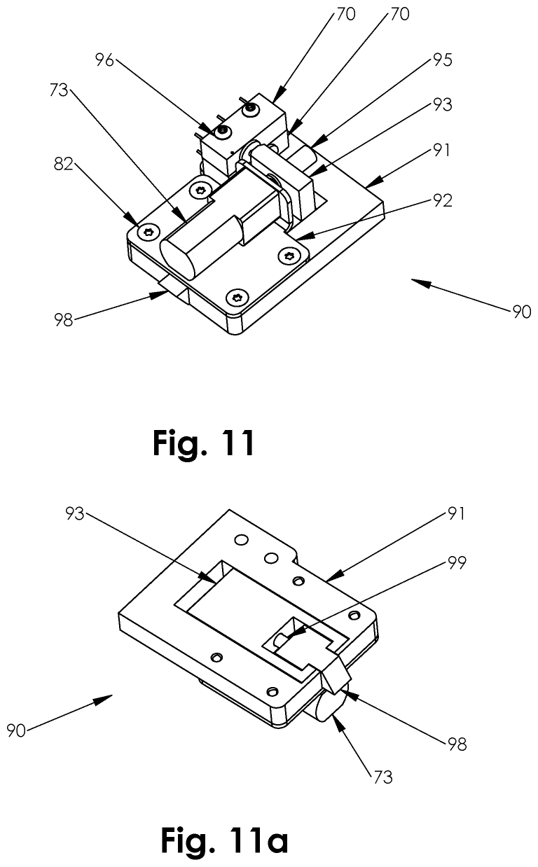

Said load door latch module is activated by a nurse's command on said tablet computer via said tablet computer interface and said electronic motor driver to provide means of opening said load door for loading personal medication containers into the device. As further explained and shown in FIG. 10 and FIG. 10a, the load door latch module retracts the latch 98, thereby providing access to empty carriage cups 30.
Said carriage chain drive module is activated by a nurse's command on said tablet computer via said tablet computer interface and said electronic motor driver. As further explained in FIG. 11 and FIG. 11A, said carriage chain drive module rotates the carriage chain 46 one full cycle permitting said bar-code reader module to read the bar-code 24 on each said personal medication container that is present and load the information from said bar-codes into said tablet computer. When a nurse is ready to dispense a said personal medication container, said tablet computer via the computer interface and the motor driver, advances the carriage cup chain until the desired said personal medication container is confirmed by said bar-code reader module. At this time the optical sensor 116 signals the tablet computer to stop said carriage chain drive module in the exact position for dispensing the selected said personal medication container. The bar-code reader module 120 reads the bar-code from the top of the personal medication container to verify that the correct said personal medication container is in the dispense location.

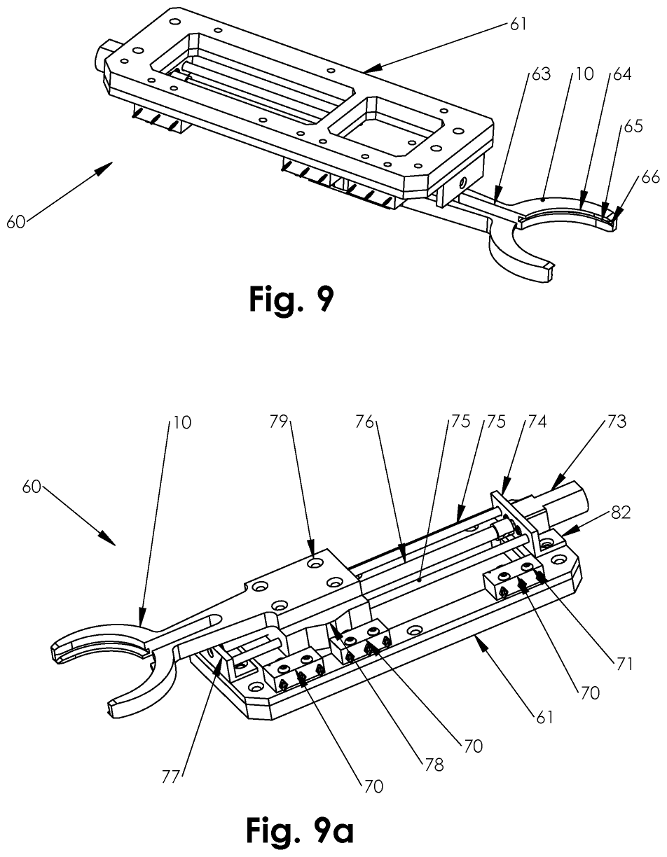

As further explained in FIG. 9 and FIG. 9a, said dispense arm module is the activated by said tablet computer via said tablet computer interface and said electronic motor driver. Said dispense arm is driven forward to press a personal medication container against said dispense door which in turn forces the personal medication container into said dispense arm. Said dispense arm is stopped until said dispense door module opens said dispense door permitting said dispense arm to move an additional amount thereby moving said personal medication container to a position accessible by a nurse. Upon command from said tablet computer, said dispense arm retracts and said dispense door closes.

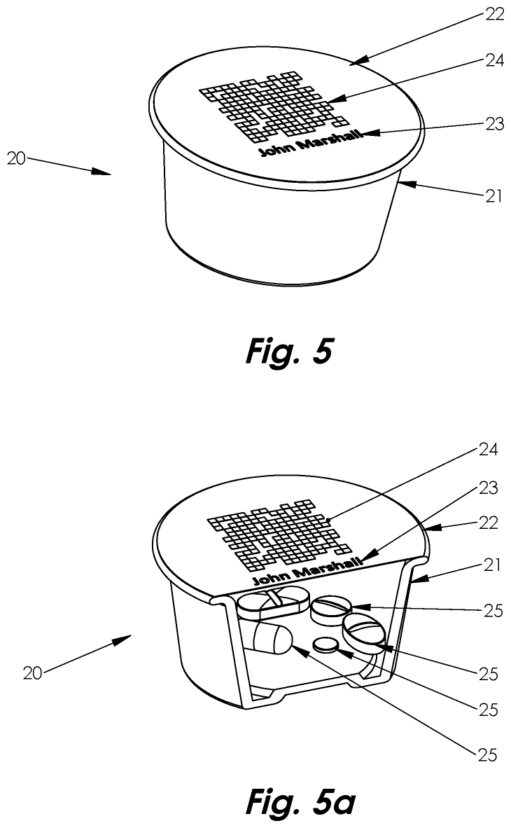

FIG. 5 shows a possible configuration of said sealed personal medication container ready for use. The container 21 is a vacuum formed plastic cup that is 1.8 inches diameter and 0.9 inches high, similar to a ¾ ounce Solo cup model P075S but may vary to accommodate various pill quantities and pill loading systems. The container may also be a blister pack container. The medication cup cover 22 is a flat liquid proof material that is sealed to said medication cup to prevent tampering and spills. The material of said medication cup cover is such that it can be readily punctured my a nurse to provide access to the medication 25 contained within. The patient's name 23 and a data-matrix bar-code 24 may be printed on the outer surface of said medication cup cover which may contain the patient's name, patients' hospital identifier number, time and date to be dispensed, and said medications which should be in said personal medication container. Other identifying methods such as alpha numeric text or an RFID chip may be used to identify said personal medication containers.

FIG. 5a shows said personal medication container shown in FIG. 4 comprised of said medication cup with a portion cut away to display the open volume and medications that may be contained for a single pour.

FIG. 6 shows an individual carriage cup 30. said carriage cup is configured in a “C” shape 33 such that it will lightly retain a said personal medication container regardless of the orientation of the invention without doing damage to said personal medication container yet a nurse may put a said personal medication container in it and said dispense arm may remove said personal medication container in a sliding motion. Said carriage cup has a lower surface 32 to further support the personal medication container and an arm 34 to provide an upper restraint to the personal medication container. A small surface conforms to said personal medication container to facilitate removing said personal medication container from said carriage cup is fabricated from a naturally slippery material for sliding and is generally 0.10 inches larger than said medication cup to allow for good injection molding practices.

FIG. 6a is similar to FIG. 5a but shows the lower side of said carriage cup. said carriage cup is further comprised of two wear bars 37 slightly higher than the lower surface 36 to slide on a guide track and two holes 38 to accept pins that may be pressed into said carriage cup.

FIG. 6b shows an individual carriage cup module 45 comprised of a said carriage cup, two carriage bushings 39, two carriage pins 42, and a carriage link 40. said carriage pin is generally stainless steel. 125 inches diameter with a 0.25 diameter by 0.06 inch high head but may vary. said carriage bushing is generally 0.13 inches inside diameter by 0.24 inches outside diameter by 0.14 inches tall but may vary. said carriage link is generally made from rigid plastic and is 0.125 thick and has two 0.13 diameter holes 41 separated by 0.5 inch but may vary. Most generally, fourteen said carriage cup assemblies are linked together in the invention by inserting said carriage pin through said carriage link of the next carriage cup assembly to form a chain.

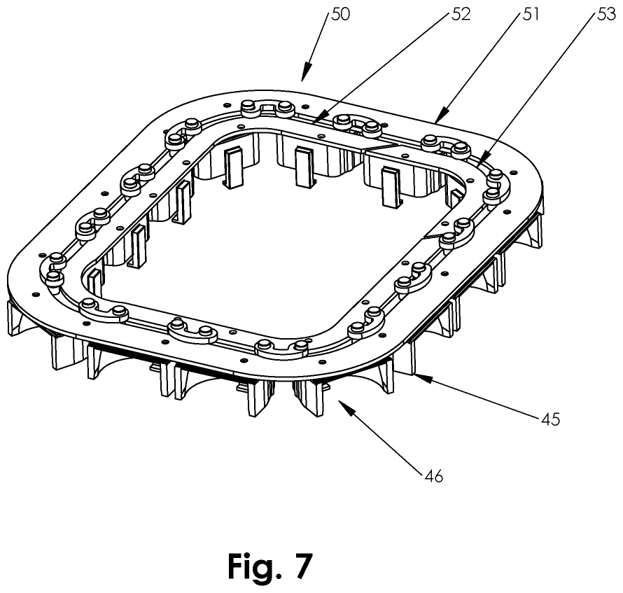

FIG. 7 shows fourteen said carriage cup modules arrange into the carriage chain 46 which is contained within the slot as defined by said track guide module. The actual number of carriage cups assemblies may vary depending on the actual size of cup, the path of the guide track, and size of the enclosure. During assembly of this device, the outer track guide 51 is affixed to said lower enclosure via screws through the track guide mounting screw holes 54. Said carriage chain is positioned within said outer track guide. The main inner track guide 52 is then positioned within said carriage chain and affixed to said lower enclosure. The secondary track guide 53 can then be moved into position and affixed to said lower enclosure thereby entrapping said carriage chain within the path defined by the track guide module 50.

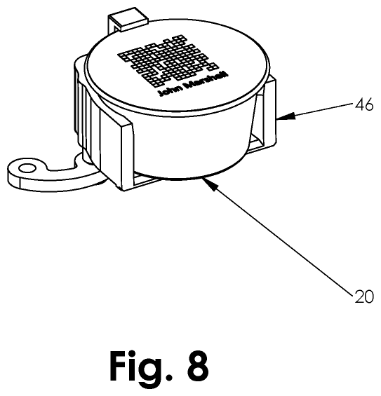

FIG. 8 shows said personal medication container secured in said carriage cup module.

FIG. 9 and FIG. 9a show the individual components of dispense arm module 60. Said dispense arm is mounted to slide arm block 78 which has two through holes to accommodate arm shafts 75 which are constrained by arm shaft mount 77 and gear motor mount 74. Said arm slide block also had a threaded opening to accommodate the threaded arm lead screw 76. Upon command from the tablet computer, the miniature gear motor 73 which is mounted to said rear motor mount 74 rotates said arm lead screw to move said arm slide block and said dispense arm from a retracted position to an intermediate position where said personal medication container is forced past the dispense arm chamfer 66 and dispense arm lead in 63 into the dispense arm retainer ring 64. Said dispense arm can then be moved further to a dispense location. The retracted, intermediate position, and dispense location are defined by three switches 70 mounted to dispense arm base 61 by limit switch screws 71. Said dispense arm has a slot 63 to allow it to not interfere with said carriage upper retainer. Said dispense arm base is located and bonded to said upper enclosure.

FIG. 10 shows the individual components of dispense door module 80. Said dispense door to has a through hole to accommodate dispense door rail 84 which is constrained by pockets in dispense door base 81 and retained by two screws for plastic 82 and a threaded opening 87 to accommodate the threaded dispense lead screw 86. Upon command from said tablet computer, said miniature gear motor 73 which is mounted to dispense motor mount 85 rotates said dispense lead screw to move said dispense door from an open to a closed position. Said dispense arm can then be moved further to a dispense location. The open and closed locations are defined by two switches 70 mounted to said dispense door base said limit switch screws 71. Said dispense door base is located and bonded to said lower enclosure.

FIG. 11 and FIG. 11a show the individual components of load door latch module 90. Said load door is released from its locked state when the load door latch 98 is retracted Upon command from said tablet computer, said miniature gear motor 73 which is mounted to load door motor mount 92 rotates load door lead screw to move load door slide plate 93 thereby moving said load door latch so that it permits said load door to be opened. Said load door motor mount is mounted to load door base 91 which guides said load door slide plate by said screws for plastic 82. After a few seconds, upon command from said tablet computer, said miniature gear motor rotates in the opposite direction to retract said load door slide plate. At this time, when said load door is moved to the closed and locked position, said load door latch move to allow said load door to pass and compresses load door latch spring 99 permitting said load door latch to retract momentarily to allow the load door to close and lock. The open and closed locations of said load door slide plate are defined by two switches 70 mounted to said dispense door base two limit switch screws 96 and activated as the load door slide plate moves past. Said load door base is located and bonded to said upper enclosure.

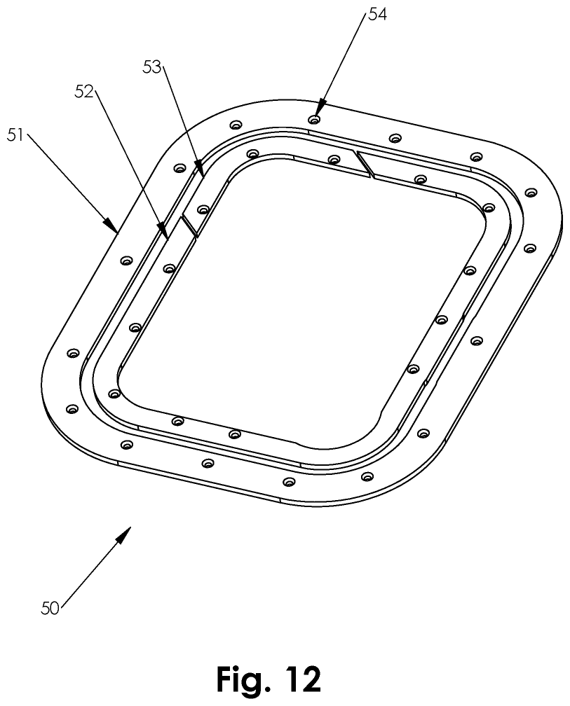

FIG. 12 and FIG. 12a show the individual components of chain drive module 100. Upon command from said tablet computer, the chain drive motor 102 mounted to the chain drive motor mount 101 rotates thereby driving primary gear 103 causing the idler gear 104 located on the idler gear shaft 109 to rotate thereby causing the driven gear 105 to rotate. Said chain drive motor mount is generally a rigid material such as aluminum and is attached to said chain drive motor by four motor mounting screws 108. Said driven gear has the chain sprocket 106 and sensor ring 107 attached to it by four said screws for plastic. Said sensor ring is an opaque material with five openings 110 for detection by the optical sensor 116 and four adjustment slots 111 for fine adjustment of said sensor ring. Said optical sensor in mounted to the sensor mount 117 with two said screws for plastic which in turn can be affixed to said lower enclosure. Upon a signal from said optical sensor, said tablet computer stops said chain drive motor in the proper position for dispensing said personal medication container. The said optical sensor may be a Honeywell 4456 part.

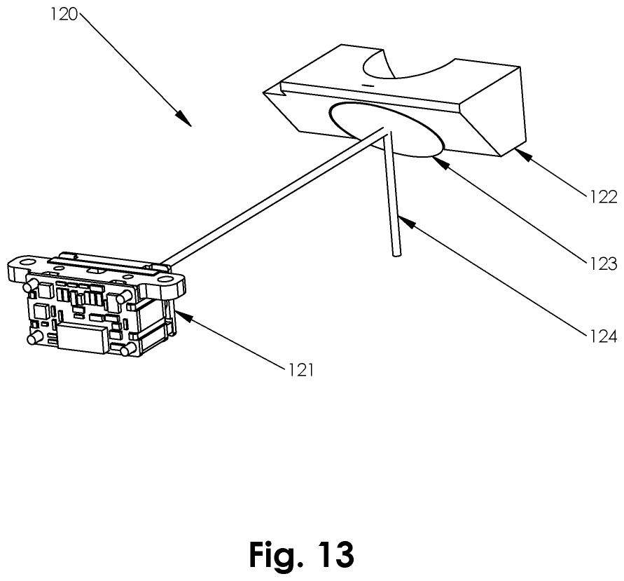

FIG. 13 shows the components of said bar code reader module. The bar code reader 121 shines the bar code reader beam 124 at the bar code mirror 123 mounted in mirror mount 122. Said bar code reader beam is deflected by said bar code mirror so shine directly on said bar code on the top of said personal medication container. Said bar code reader is generally a model Md143 manufactured by Matrix. Other bar code readers may be used as necessary. Both said bar code reader and mirror mount are located and bonded to said upper enclosure.

### BRIEF SUMMARY OF THE INVENTION

Referring to the drawings, a device for easily transporting and dispensing prepackaged medication containers about the size of a standard ream of paper for is disclosed. The device may be larger or smaller depending on the quantity or shape of the personal medication containers used. The device may have 14 container holders for prepackaged medication containers which is a higher than average number of patients to be on a nurses care on a hospital wing. The devise may have an arm that moves to remove a single prepackaged medication container from the container holder and present it to a nurse.

Further disclosed is a secured device with one or more locked doors for loading and dispensing the prepackaged medication containers. The container holders may be arranged and linked in a chain configuration and driven in a serpentine path with its exact location identified by a optical sensor reading a slotted disk on the chain drive sprocket so that a specific cup holder may be positioned at the load door or at the dispense door.

Further disclosed is a device that has a computing device with a touch screen that controls the internal functions of the devise such as operating motors to move the cup holder chain and motors to open and close doors and may interface with a hospital computer network. The computing device and touch screen may also be used to record patient vital information while the nurse is at a patient's bedside and transmit this data to a hospital computer system. The internal function of the devise may be powered by a rechargeable battery securely mounted within the device.

The device may have a bar-code reader to scan and read bar-codes on the upper surface of the personal medication containers for accurately identifying each personal medication cup.

The device may utilize a shoulder strap for hands free carrying of the device from one location to another.

The device may have a flat area adjacent to the touch screen for use as a writing surface to allow a nurse to take patient notes on paper.

1. A secure portable device consisting of an enclosure for delivering multiple prepackaged personal mediation containers to a patient providing access to only a user selected personal medication container upon demand comprising:
a locked door or doors for loading and accessing personal medication containers,
a touch screen for controlling the device,
an internal mechanism for accessing a specific personal medication container,
a means of automatically identifying each personal medication container,
1. A portable device according to claim 1 which has motorized locks on all doors.
2. A portable device according to claim 1 which has a shoulder carrying strap.
3. A portable device according to claim 1 which interfaces with a hospital WiFi system.
